(19) United States
(12) Patent Application Publication
(10) Pub. No.: US 2021/0391042 A1
(43) Pub. Date: Dec. 16, 2021

(54) MANAGEMENT SYSTEM

(71) Applicant: FUJIFILM Corporation, Tokyo (JP)

(72) Inventor: Yasuhisa KANEKO, Ashigarakami-gun (JP)

(73) Assignee: FUJIFILM Corporation, Tokyo (JP)

(21) Appl. No.: 17/459,012

(22) Filed: Aug. 27, 2021

Related U.S. Application Data
(63) Continuation of application No. PCT/JP2020/000651, filed on Jan. 10, 2020.

(30) Foreign Application Priority Data
Feb. 28, 2019 (JP) 2019-036591

(51) Int. Cl. G16H 10/40; G16H 40/20; G06K 7/14; G06T 7/90; H04N 5/232; G06T 7/00

(52) U.S. Cl. CPC G16H 10/40; G16H 40/20; G06K 7/1417; G06T 2207/10024; H04N 5/232; G06T 7/0012; G06T 2207/30004; G06T 7/90

(57) ABSTRACT

A management system includes a test order acquisition unit that acquires a test order, a communication unit that receives a subject identifier, container information of a sample container used by a subject for storing a sample, and a container image which is an image of the sample container from a subject terminal through communication with the subject terminal, a determination unit that determines a success or failure of sample collection by using the container image, and a registration unit that registers the test order, the container information, and a determination result in the determination unit in association with each other by using the subject identifier.

### CROSS-REFERENCE TO RELATED APPLICATIONS

This application is a Continuation of PCT International Application No. PCT/JP2020/000651 filed on 10 Jan. 2020, which claims priority under 35 U.S.C § 119(a) to Japanese Patent Application No. 2019-036591 filed on 28 Feb. 2019. The above application is hereby expressly incorporated by reference, in its entirety, into the present application.

### BACKGROUND OF THE INVENTION

### 1. Field of the Invention

The present invention relates to a management system that manages a subject, a test result, a test order that is a request for a test, and the like in association with each other.

### 2. Description of the Related Art

In the related art, various tests such as a fecal occult blood test or a blood test have been performed as needed for a health diagnosis and the like. Some of these tests are performed at a hospital, but the hospital usually requests a testing institution to perform a test. That is, the testing institution that actually performs the test may be a different institution from the hospital that uses test results.

For example, in the case of a fecal occult blood test, a subject collects a sample such as feces and submits it to a hospital or a testing institution. On the other hand, a doctor or the like in a hospital specifies a subject to the testing institution and requests the testing institution to perform a fecal occult blood test. Then, the testing institution performs a test on a sample of the subject or a specific subject provided by the hospital based on a test order received from the hospital, and notifies the hospital and/or the subject of the result.

Then, usually, the testing institution is manually performing the work of associating the test result related to the sample in a sample container containing the sample with the test order. In recent years, in a case where an ID of a subject and a test result are output in association with each other from a test device and the subject is registered in a test order with the same ID, a management device that automatically associates the test result with the test order is known (JP2018-084862A). In addition, in systems used in private hospitals and the like, there is also known a system that specifies a subject to be tested next and automatically registers a test result output by a test device as a test result of the specified subject (JP2011-206557A).

### SUMMARY OF THE INVENTION

In tests such as a fecal occult blood test, it is necessary to correctly collect and store a sample to the extent that the test can be normally performed. However, in a test in which a subject collects and submits a sample by himself/herself, the subject may submit a sample container in which the sample is not correctly collected or stored. In this case, as a matter of course, accurate test results cannot be obtained, which is disadvantageous to the subject in the end. In particular, even though the subject intends to correctly collect the sample according to the instruction manual or the like attached to a test container, it is a burden for the subject to be forced to retest because accurate test results cannot be obtained as a result. Therefore, there is a demand for registering and managing whether or not the sample can be correctly collected and stored before the test is performed.

In addition, in a case where a testing institution performs a test on a sample that has not been collected and stored correctly, a test device usually outputs an error to that effect. Therefore, employees of the testing institution have to deal with the error output by the test device, and the fact that the sample is not correctly collected and stored is one of the causes of the workflow delay. Therefore, even in the testing institution, there is a demand for registering and managing whether or not the sample can be correctly collected and stored before the test is performed.

An object of the present invention is to provide a management system for registering and managing that a sample is correctly collected and stored before a test is performed.

According to an aspect of the present invention, there is provided a management system comprising a test order acquisition unit, a communication unit, a determination unit, and a registration unit. The test order acquisition unit is configured to acquire a test order including a subject identifier that specifies a subject terminal used by a subject, the communication unit is configured to receive the subject identifier, container information, and a container image from the subject terminal through communication with the subject terminal, the container information being information related to a sample container used by the subject for storing a sample, and the container image being an image of the sample container, the determination unit is configured to determine a success or failure of sample collection by using the container image, and the registration unit configured to register the test order, the container information, and a determination result in the determination unit in association with each other by using the subject identifier.

It is preferable that the subject identifier is an e-mail address or a telephone number used by the subject.

It is preferable that the container information includes at least a container identifier that specifies the sample container.

It is preferable that the sample container has a transparent portion in which the sample is stored and/or a transparent portion through which the sample passes, and the container image is an image including the transparent portion of the sample container.

It is preferable that the determination unit determines the success or failure of the sample collection based on a color and/or a density of the transparent portion.

It is preferable that the determination unit determines that the collection of the sample is successful in a case where the sample or a portion containing the sample has a specific color and a density equal to or higher than a specific density, and determines that the collection of the sample fails in a case where the sample or the portion containing the sample does not have the specific color or in a case where the sample or the portion containing the sample has a density lower than the specific density.

It is preferable that the sample container has a two-dimensional code including the container information, and the subject terminal acquires the container information from the two-dimensional code and transmits the container information to the communication unit.

It is preferable that the two-dimensional code includes a control command for controlling the subject terminal.

It is preferable that the control command includes an information transmission command for transmitting the container information and the container image to the communication unit.

It is preferable that the control command includes a camera activation command for activating a camera that captures the container image.

It is preferable that the communication unit receives the container information and a test result from a test device that tests the sample, and the registration unit registers the test result in association with the test order by using the container information.

With the management system according to the aspect of the present invention, it is possible to register and manage that the sample is correctly collected and stored before the test is performed.

### BRIEF DESCRIPTION OF THE DRAWINGS

FIG. 1 is an explanatory diagram showing an outline of a test such as a fecal occult blood test.

FIG. 2 is a sample container for a fecal occult blood test.

FIG. 3 is an external view of a fecal occult blood test device, which is a test device.

FIG. 4 is a block diagram showing a configuration of a test device and a management system.

FIG. 5 is an example of a table in which a test order, a test result, and the like are associated with each other.

FIG. 6 is a flowchart showing a flow of registration of data and the like in a management system.

FIG. 7 is a flowchart relating to registration of a test order.

FIG. 8 is an explanatory diagram showing the contents of a table after acquisition of a test order.

FIG. 9 is a flowchart showing a flow of registration of subject input information and container information.

FIG. 10 is a table in which subject input information and container information are registered.

FIG. 11 is a flowchart showing a flow of registration of tester information and test results.

(A) and (B) of FIG. 12 are modification examples of a sample container.

FIG. 13 is a modification example of a code attached to a sample container.

### DESCRIPTION OF THE PREFERRED EMBODIMENTS

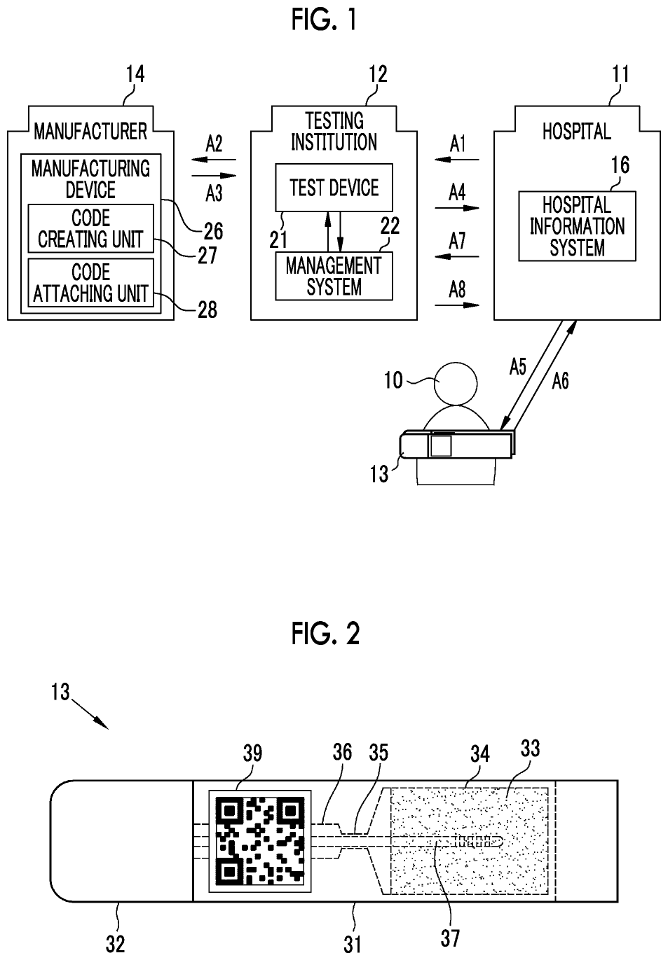

As shown in FIG. 1, in various medical-related tests such as a fecal occult blood test or a blood test (hereinafter, referred to as a “test”), a hospital 11 visited by a subject 10 for a health diagnosis, other tests, or diagnosis, a testing institution 12 that performs the test upon request from the hospital 11, a manufacturer 14 that manufactures a sample container 13 for collecting and/or storing the sample related to the test, and the like are usually involved. The hospital 11 visited by the subject 10 for a health diagnosis or the like includes an institution that specializes in health diagnoses such as the Occupational Health and Welfare Association that travels to companies or local governments, or an institution (company) that provides test services for the general public.

Specifically, in a case where a test is required, the hospital 11 issues a request for the test (hereinafter referred to as a test order) to the testing institution 12 (Step A1). In a case where the test order is acquired, the testing institution 12 orders the manufacturer 14 for the sample container 13 for collecting and/or storing the sample (Step A2). The sample is the body tissue or secretions of the subject 10. For example, in the fecal occult blood test, the sample is the feces of the subject 10. In the blood test, the sample is the blood of the subject.

In a case where the manufacturer 14 delivers the sample container 13 corresponding to the order from the testing institution 12 to the testing institution 12 (Step A3), the testing institution 12 sends the sample container 13 to the hospital 11 (Step A4). Then, the hospital 11 sends the sample container 13 and a guide or the like describing a method of collecting the sample to the subject 10 (Step A5). Upon receiving the sample container 13 and the like, the subject 10 collects the sample in the sample container 13 by himself/herself and submits the sample container 13 in which the sample is stored to the hospital 11 (Step A6). In a case where the hospital 11 receives the sample container 13 in which the sample is stored from the subject 10, the hospital 11 transfers the sample container 13 to the testing institution 12 (Step A7), and the testing institution 12 performs the test. After that, the testing institution 12 reports the test result to the hospital 11 in association with the test order (Step A8). The manufacturer 14 can also manufacture the sample container 13 in advance without waiting for an order from the testing institution 12.

For example, the hospital 11 issues a test order to the testing institution 12 by using a hospital information system (HIS) 16. The test order includes at least requester information such as the surname or the name that specifies a requester of the test (issuer or issuing institution of the test order) and other ID or e-mail address, subject information which is information related to the subject 10 who performs the test, and information indicating the target (sample) and content (test content) of the test to be performed. In addition, the subject information includes a subject identifier in addition to the name, gender, age, and the like of the subject. The subject identifier is an identifier (ID (identification)) that can specify the individual of the subject 10, and is a character string including characters, symbols, and the like. The subject identifier included in the test order is, for example, a character string (so-called patient ID) given by the hospital 11 to a specific subject 10 for management, an e-mail address of the subject 10, a telephone number (particularly a mobile phone number that functions as an account for a short message service (SMS), etc.), a social network service (SNS), other account information (account name and the like) related to an individual-specifying service, or a combination thereof.

In particular, the test order includes at least one subject identifier that can specify the terminal used by the subject (hereinafter referred to as a subject terminal). The subject identifier that can specify the subject terminal is, in the above example, an e-mail address, a mobile phone number, or the like. In the present embodiment, it is assumed that the test order includes the e-mail address of the subject 10 as the subject identifier that specifies the subject terminal. The subject terminal is, for example, a smartphone, a tablet terminal, a mobile phone, a personal computer, a smart speaker, or other electronic devices, and is a smartphone in the present embodiment.

The testing institution 12 comprises a test device 21, and a management system 22. The test device 21 that performs the test acquires a sample from the sample container 13 submitted by the subject 10 and performs the test according to the test order. The management system 22 manages a test order, subject information, and a test result output by the test device 21 in association with each other.

In particular, the management system 22 of the present embodiment determines a success or failure of collection of the sample (hereinafter referred to as a success or failure of the sample collection or a sample collection success/failure) in addition to the above-mentioned association, and manages the determination result in association with the above-mentioned test order or the like. The success or failure of collection of the sample refers to whether or not the collection and/or storage of the sample is correct enough to obtain accurate test results. In a case where the sample is correctly collected and stored, the success or failure of the sample collection is “normal” (or “successful”). In a case where the sample is not correctly collected or stored, the success or failure of the sample collection is “abnormal” (or “unsuccessful”).

Further, the management system 22 communicates with the hospital information system 16 of the hospital 11, the system of the manufacturer 14, and/or the subject terminal, etc., as necessary. That is, the management system 22 can not only manage the test results and the like in the testing institution 12, but also acquire the test order, place the order for the sample container 13, acquire the subject information, report the test results, and the like. The management system 22 is composed of one or a plurality of computers and the like. Further, one or a plurality of computers and the like constituting the management system 22 can be partially or wholly installed outside the testing institution 12. Further, the management system 22 may be a management system 22 configured in the form of a service provided by a computer or the like installed outside the testing institution 12 (for example, the form such as a cloud service).

The testing institution 12 may have one or a plurality of types of test devices 21 depending on the type of test to be performed. In a case where the testing institution 12 supports a fecal occult blood test and a blood test, the testing institution 12 comprises two types of test devices 21, a fecal occult blood test device and a blood test device. Further, the test device 21 may have a plurality of test devices 21 used for the specific test in order to perform the specific test substantially at the same time. For example, the testing institution 12 can have a plurality of fecal occult blood test devices, which are test devices 21.

The manufacturer 14 comprises a manufacturing device 26 for manufacturing the sample container 13. The manufacturing device 26 comprises a code creating unit 27 and a code attaching unit 28. The code creating unit 27 creates a code 39 (see FIG. 2) to be attached to the sample container 13, and the code attaching unit 28 attaches the code 39 created by the code creating unit 27 to the manufactured sample container 13. The code is, for example, a one-dimensional barcode, a two-dimensional code (so-called quick response (QR) code (registered trademark)), or the like.

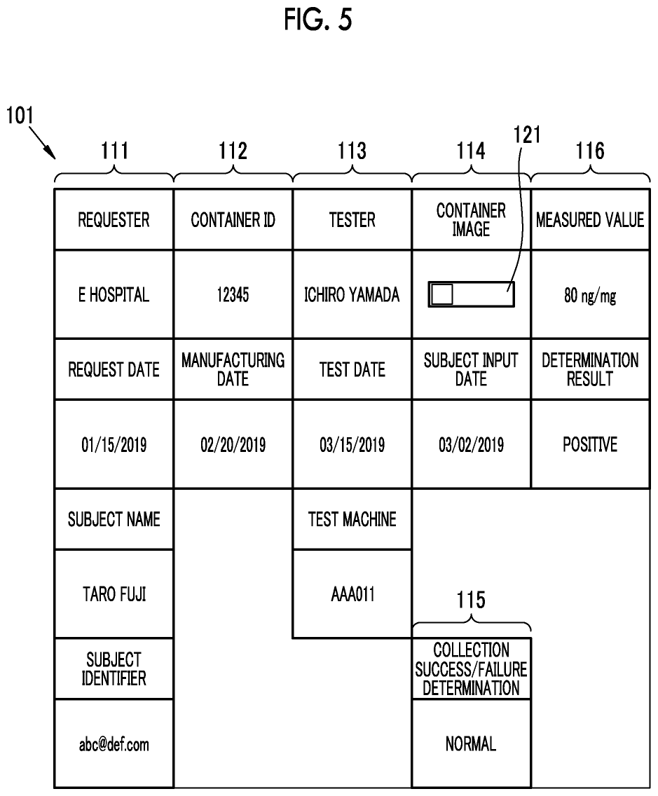

The code 39 attached to the sample container 13 includes at least a container identifier (for example, product ID or serial number) that identifies the individual of the sample container 13, information that specifies the product type, the production lot, and/or the manufacturing date, or information for accessing the acquisition source of such information (hereinafter, referred to as container information 112, see FIG. 5). Further, the code 39 can include a control command for a device that reads the code 39 or another device connected to the device that reads the code 39. The control command for a device refers to a character, a symbol, a signal, or the like that puts the device into a state in which a specific function is performed by using a part or the whole of the device. The specific function is, for example, activating a designated application, sending designated information, or the like.

In the present embodiment, the code creating unit 27 creates a two-dimensional bird-shaped code 39 including a control command for the subject terminal, and the code attaching unit 28 attaches the code to each sample container 13. Therefore, in a case where the device that scans the code 39 by reading the code 39 (hereinafter referred to as scanning) can acquire the container information 112 of the sample container 13 and scans the code 39 using the subject terminal, the subject terminal performs a specific operation according to the control command included in the code 39. Further, in the present embodiment, the code 39 includes an information transmission command for transmitting the container information 112 and a container image 121 (see FIG. 5) to a communication unit 63 (see FIG. 4) at the subject terminal, and a camera activation command for activating a camera that captures the container image 121 (or an application that controls the camera).

Hereinafter, it is assumed that the test to be performed is specifically a fecal occult blood test. Therefore, the sample is the feces of the subject 10, and the sample container 13 is a container (a stool collection container) made into a kit so that the subject 10 can collect stool by himself/herself and store it stably for a certain period of time. The test device 21 is a fecal occult blood test device.

As shown in FIG. 2, the sample container 13 comprises a container body 31 for containing feces as a sample, and a cap 32 attachably and detachably provided on the container body 31. Inside the container body 31, a storage chamber 34 containing a storage solution 33 (or a diluent) for immersing and storing feces, and an insertion path 36 that is inserted into the storage chamber 34 via a scraping portion 35 are provided. Further, the cap 32 is integrally provided with a stool collection rod 37. The stool collection rod 37 is a portion for collecting feces as a sample, and feces can be collected by removing the cap 32 from the container body 31 and rubbing the vicinity of the tip of the stool collection rod 37 against the feces. After collecting the feces, the cap 32 is attached to the container body 31 while passing the stool collection rod 37 through the insertion path 36. Thereby, the tip portion of the stool collection rod 37 to which the feces are attached enters the storage chamber 34 through the scraping portion 35. The scraping portion 35 scrapes off excess feces in a case where it is attached to the tip portion of the stool collection rod 37. As a result, a predetermined amount of feces is immersed in the storage solution 33 and stored. The storage chamber 34 is a portion for storing the sample in the sample container 13. The scraping portion 35 is a portion through which the sample passes in the sample container 13.

In the container body 31, at least a part of the scraping portion 35 and/or the storage chamber 34 is transparent. At least a part being transparent refers to having light transmittance that allows the presence or absence of contents (or deposits, etc.) and its color to be recognized from the outside of the container body 31, and includes a case where it is translucent or a case where it has some color (for example, the color of the resin forming the container body 31). In the present embodiment, the entire container body 31 is almost colorless and transparent.

The code 39 created by the code creating unit 27 is attached to, for example, one surface of the container body 31. Here, the attachment position of the code 39 exposes at least the scraping portion 35 and/or at least a part of the storage chamber 34. This is to confirm the success or failure of sample collection in the scraping portion 35 and/or the storage chamber 34. In the present embodiment, the code 39 is attached to the cap 32 side of the container body 31 to expose the entire scraping portion 35 and the entire storage chamber 34.

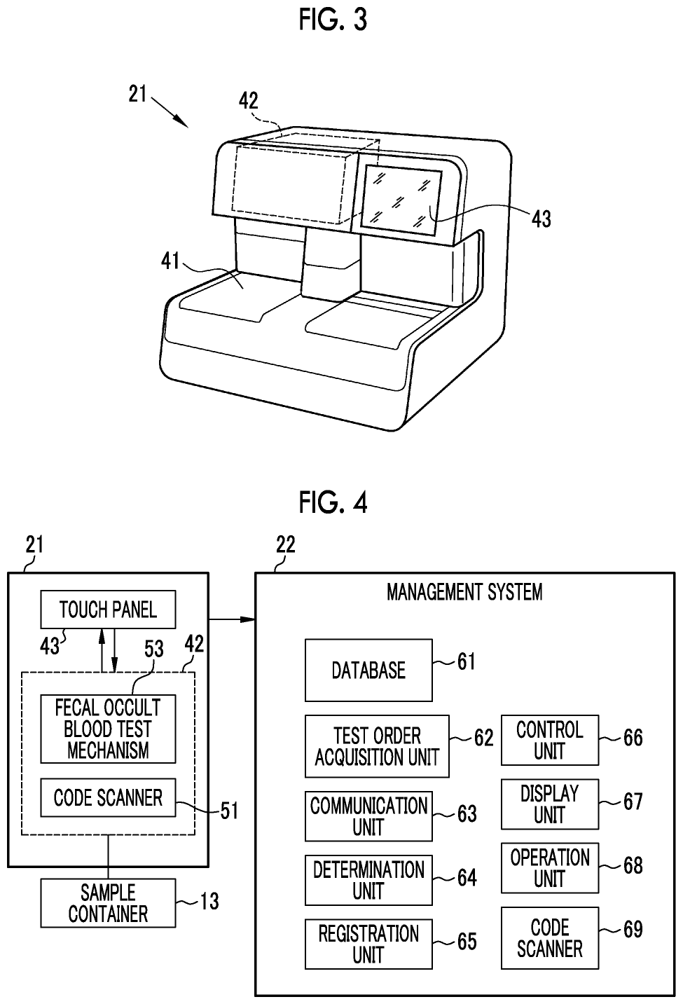

As shown in FIG. 3, the test device 21 is a fecal occult blood test device, and comprises a sample container placing portion 41, a test unit 42 which is a substantive mechanism for performing a test, and a touch panel 43 which functions as an operation unit and a display unit of the test device 21. The sample container placing portion 41 is a portion on which one or a plurality of sample containers 13 are placed in a case of performing a test.

As shown in FIG. 4, the test unit 42 comprises a code scanner 51 (reading unit) and a fecal occult blood test mechanism 53. The code scanner 51 acquires the container information 112 by scanning the code 39 attached to the sample container 13.

The fecal occult blood test mechanism 53 is a mechanism for performing a fecal occult blood test, and includes a stock of reagents used for the fecal occult blood test. In a case where the test is completed, the fecal occult blood test mechanism 53 sequentially outputs the container information 112 and the test result to the touch panel 43 and the management system 22. The touch panel 43 indicates the progress of the test by appropriately displaying the test result or the like in the order in which the test is completed. Further, in a case where the test result or the like is acquired from the fecal occult blood test mechanism 53, the management system 22 associates the test result or the like with the test order and registers and manages the data.

The management system 22 comprises a database 61, a test order acquisition unit 62, a communication unit 63, a determination unit 64, a registration unit 65, a control unit 66, a display unit 67, an operation unit 68, a code scanner 69, and the like.

The database 61 stores the test order 111 and the test result or the like in association with each other. The data held by the database 61 is, for example, in the format of a table 101 shown in FIG. 5. The table 101 includes a test order 111, a container information 112, tester information 113, information related to the input from the subject (hereinafter referred to as subject input information) 114, a determination result 115 of sample collection success/failure, and a measurement result and other information related to the test result (hereinafter referred to as a test result) 116.

The test order 111 held in the table 101 is a part or all of the test order acquired from the hospital information system 16 or the like. In the present embodiment, in the table 101, the name of the “requester” which is requester information, the “request date” of the test, the “subject name” which is the name of the subject, and the subject identifier are registered and managed in a form that can be used in the management system 22. In FIG. 5, the “requester” is “E hospital” and the “request date” is “Jan. 15, 2019”. The “subject name” is “Taro Fuji”, and the subject identifier is the e-mail address “abc@edf.com” used by the subject 10 on the subject terminal or the like.

The container information 112 held in the table 101 is a part or all of the container information that can be acquired via the code 39 attached to the sample container 13. In the present embodiment, in the table 101, the “container ID” which is the container identifier of the sample container 13 and the “manufacturing date” thereof are registered and managed. In FIG. 5, the “container ID” is “12345” and the “manufacturing date” is “Feb. 20, 2019”.

The tester information 113 held in the table 101 refers to information related to a person or an object related to the execution of the test, and is, for example, the contents of each item of a “tester” which is the name or ID of the person who performed the test (for example, the person in charge of operating the test device 21 or the person in charge of the test (hereinafter referred to as the tester)), a “test date” which is the date at which the test is performed, and/or a “test machine” which is the information which specifies the test device 21 used for performing the test. In FIG. 5, the “tester” is “Ichiro Yamada”. The “test date” is “Mar. 15, 2019”, and the “test machine” is “AAA011” which is an identification code of the test device 21.

The subject input information 114 held in the table 101 refers to information related to the subject 10 or the sample container 13 used by the subject 10 to be registered and managed by receiving the information provided by the subject 10, and is, for example, a container image 121 which is an image obtained by capturing a part or all of the sample container 13, an input date of subject input information 114 (“subject input date”), and the like. In FIG. 5, the “subject input date” is “Mar. 2, 2019”. In the table 101, the correlation (association) between the subject name and the like and the container ID is automatically performed via the subject identifier (e-mail address or the like) of the subject 10 by reading the code 39 attached to the sample container 13 with the subject terminal (smartphone or the like) and sending an e-mail or the like to the management system 22. In the related art, the subject 10 manually writes his/her name etc. on the label attached to the sample container 13, and the tester of the testing institution 12 manually performs the work of collating the name written on the label with the corresponding table on the management system and correlating them. Compared with this, the management system 22 can easily make the above association.

The determination result 115 of sample collection success/failure held in the table 101 is “normal”, “abnormal”, or a flag indicating these. In FIG. 5, “sample collection success/failure” is “normal”.

A test result 116 held in the table 101 is a “measured value” output by the test device 21 and/or a result of determination (“determination result”) obtained by comparing the measurement result output by the test device 21 with a predetermined reference. In FIG. 5, the “measured value” and the “determination result” are registered and managed. The “measured value” is “80 nm/mg” and the “determination result” is “positive”.

The test order acquisition unit 62 acquires a test order. The test order acquisition unit 62 can acquire the test order manually or automatically. The manual acquisition of the test order means that the tester or the like inputs information into the item of the test order 111 in the table 101 by using an operation unit 68. The automatic acquisition of the test order means that the test order acquisition unit 62 connects to the hospital information system 16 or the like via, for example, the communication unit 63, obtains information on the test order from the hospital information system 16 or the like, and inputs information on the item of the test order 111 into the table 101.

The communication unit 63 is an interface for communicating with the hospital information system 16, the test device 21, the system owned by the manufacturer 14, the subject terminal, or any other device other than the management system 22, and thereby transmitting and receiving necessary information to and from the devices and the like. The communication unit 63 receives the subject identifier via the subject terminal, the container information 112 of a sample container 13 used by the subject 10 for storing a sample, and the container image 121 of the sample container 13 from the subject terminal through communication with the subject terminal, for example. Further, the communication unit 63 receives the container information 112 and the tester information 113 in a case where the test is performed, and receives the container information 112 and the test result 116 in a case where the test is completed (the test result is obtained) through communication with the test device 21.

The determination unit 64 determines the success or failure of sample collection by using the container image 121. The determination unit 64 uses the container image 121 of the sample container 13 in which a portion for storing the sample and/or a portion through which the sample passes is transparent. In addition, the determination unit 64 determines the success or failure of the sample collection based on the color and/or the density of the transparent portion in the container image 121. That is, the determination unit 64 determines that the collection of the sample is successful in a case where the sample or a portion containing the sample has a specific color and a density equal to or higher than a specific density, and determines that the collection of the sample fails in a case where the sample or the portion containing the sample does not have the specific color or in a case where the sample or the portion containing the sample has a density lower than the specific density.

In the present embodiment, the sample is the feces of the subject 10, and the color thereof is generally yellowish brown (specific color). Therefore, the colorless and transparent storage solution 33 for storing the feces as a sample usually turns light yellowish brown (specific color) after collecting the feces. In a case where the storage solution 33 contains a reagent or the like, the storage solution 33 may exhibit a color other than light yellowish brown due to the reaction of feces or components contained in feces and its reagents, but in this case as well, it is the same that it exhibits a certain color (specific color). Further, in the sample container 13 of the present embodiment, the storage chamber 34, which is a portion for storing the sample, and the scraping portion 35, which is a portion through which the sample passes, are both transparent, and the container image 121 obtained by capturing the sample container 13 includes these “transparent portions”. Therefore, in the container image 121 in which the sample container 13 is captured, the feces attached to the scraping portion 35 and the storage solution 33 in which the feces are stored can be specified. In the blood test, there is a method in which the subject 10 collects blood by himself/herself, drops the blood into a sample container containing a diluent, filters the diluent mixed with the blood with a filter, and then mails the sample to the hospital 11. In this case, in a case where the blood is not normally filtered by the filter, the diluent after filtration may be reddish. This is called hemolysis, and the test at the testing institution 12 may not be correctly performed. Therefore, it is necessary to make a determination based on color or the like in the blood test as well.

Therefore, the determination unit 64 determines the success or failure of sample collection based on the color and/or the density of the scraping portion 35 and/or the storage chamber 34 by using the container image 121. The color of the scraping portion 35 is the color of feces attached to the scraping portion 35, and the density of the scraping portion 35 is the density of the color of feces attached to the scraping portion 35. The color of the storage chamber 34 is the color of the storage solution 33 in which the feces of the storage chamber 34 are immersed, and the density of the storage chamber 34 is the density of the color of the storage solution 33 in which the feces of the storage chamber 34 are immersed.

The registration unit 65 registers the test order 111, the container information 112, and the determination result in the determination unit 64 in the database 61 in association with each other by using the subject identifier. Specifically, the registration unit 65 creates the table 101 serving as a workplace in the database 61 in a case where the test order acquisition unit 62 acquires the test order 111. Then, the contents such as the subject identifier are registered in the created table 101. In a case where the communication unit 63 receives the subject identifier and the container information 112, the registration unit 65 collates the subject identifier with the database 61 (the item of the “subject identifier” in a plurality of tables 101 included in the database 61), and specifies the table 101 in which the subject identifiers match. Then, the contents of the received container information 112 are registered in the table 101 in which the subject identifiers match. In a case where the communication unit 63 receives the container image 121 together with the subject identifier, the subject identifier is collated with the database 61 in the same manner as described above, and the container image 121 is registered in the table 101 in which the subject identifiers match. In a case where the determination unit 64 determines the success or failure of sample collection by using the container image 121, the determination result is registered in the table 101 in which the container image 121 is registered. In addition, in a case where the test device 21 performs the test, and the communication unit 63 receives the test result 116 and the like, the registration unit 65 collates the test result with the database 61 by using the container information 112 such as the container ID, and registers the test result and the like in the table 101 with which the container information 112 matches.

The control unit 66 comprehensively controls each unit of the management system 22 such as the database 61, the test order acquisition unit 62, the communication unit 63, the determination unit 64, and the registration unit 65. The display unit 67 is, for example, a liquid crystal display or the like, and the operation unit 68 is a keyboard and/or a pointing device or the like. The code scanner 69 reads the code 39. In the management system 22, the code 39 of the sample container 13 can be read manually and at any timing by using the code scanner 69.

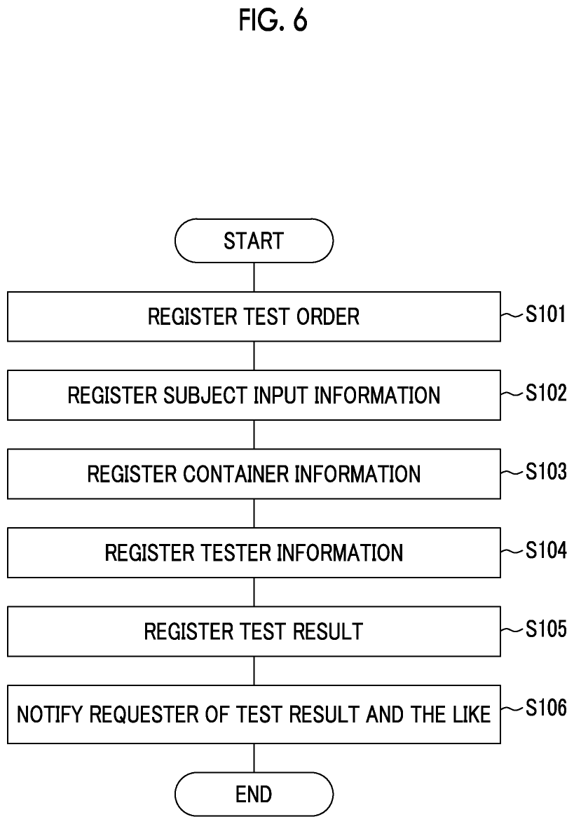

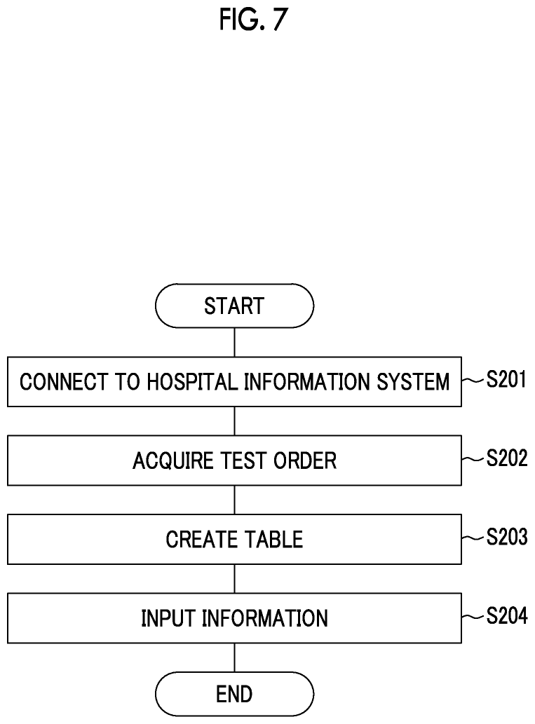

Hereinafter, the flow of operations related to registration and the like of the management system 22 configured as described above will be described. As shown in FIG. 6, the management system 22 acquires and registers a test order 111 (Step S101), acquires and registers subject information (Step S102), and acquires and registers sample container information (Step S103) before performing the test. Further, the management system 22 acquires and registers tester information (Step S104), and acquires and registers a test result (Step S105) at the time of performing the test or after performing the test. After that, the management system 22 notifies the hospital 11 or the subject 10, which is the requester, of a test result and the like (Step S106).

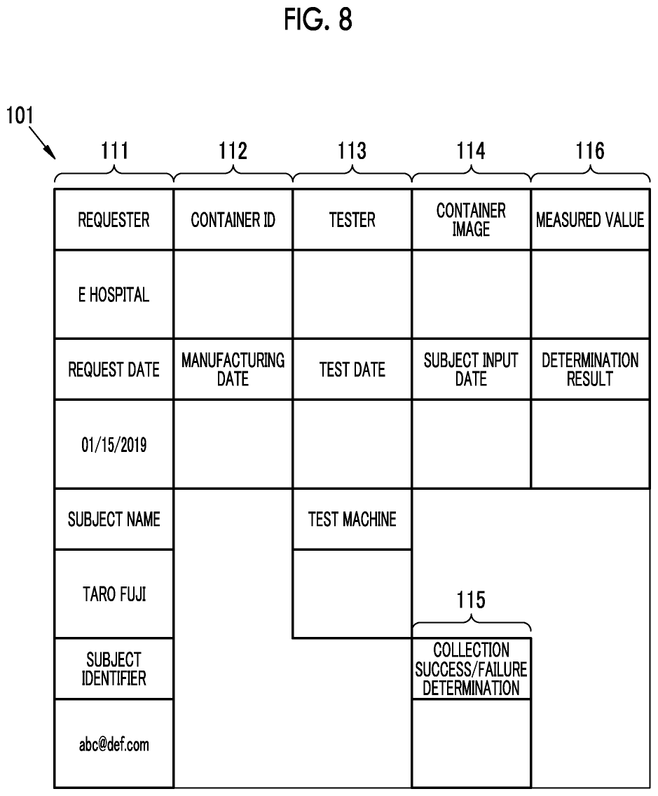

In the acquisition and registration of the test order 111 (Step S101), for example, the management system 22 first connects to the hospital information system 16 (Step S201) and acquires one or a plurality of test orders 111 (Step S202). After that, the registration unit 65 creates a table 101 in the database 61 for each test order 111 (Step S203), and inputs the items of the test order 111, “requester”, “request date”, “subject name”, and “subject identifier”. Therefore, as shown in FIG. 8, the management system 22 has a table 101 in which the items of the test order 111 are registered at the stage at which the test order 111 is acquired.

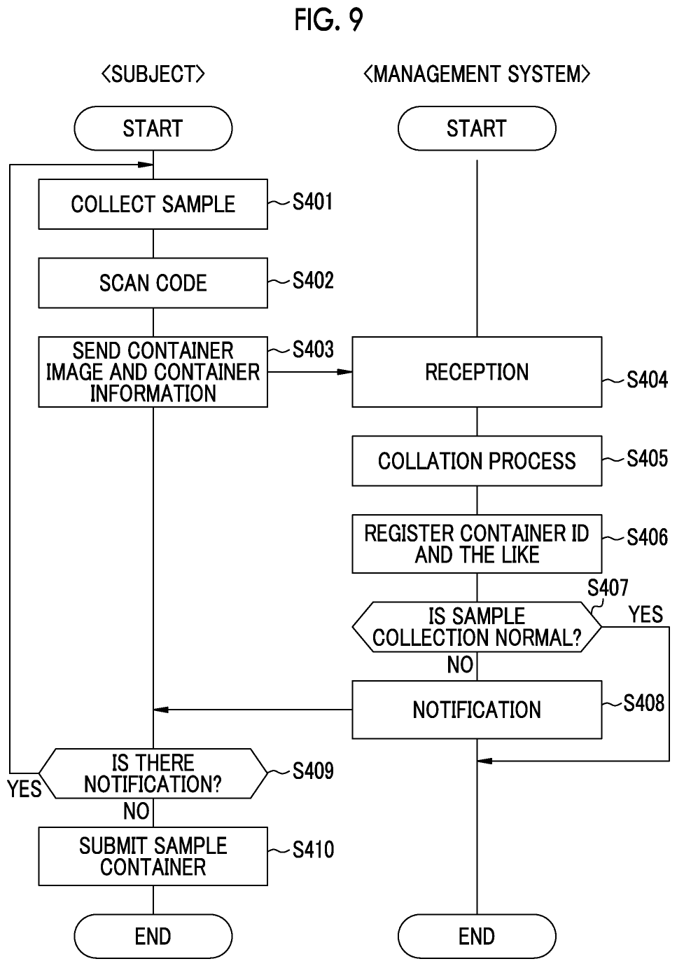

The acquisition and registration of the subject information (Step S102) and the acquisition and registration of the sample container information (Step S103) are performed as follows. That is, in a case where the subject 10 receives the sample container 13 by shipping from the hospital 11 (Step A5 in FIG. 1), as shown in FIG. 9, the subject 10 collects feces as a sample (Step S401) and stores the sample in the sample container 13. After that, the subject 10 scans the code 39 attached to the sample container 13 by using the smartphone, which is the subject terminal, according to the description of the instruction manual attached to the sample container 13 (Step S402). Since the code 39 includes a control command for driving the smartphone of the subject 10, the smartphone operates according to the control command. Therefore, the subject 10 captures the container image 121 according to the guidance of the smartphone operating according to the control command of the code 39, attaches the container information 112 thereto, and sends the container image 121 from the smartphone to the management system 22 (Step S403). Since the container image 121 is sent by using the mail function or the message function, the e-mail address, which is the subject identifier, is automatically attached and sent to the management system 22. Further, depending on the content of the control command, the smartphone, which is the subject terminal, can automatically perform these operations.

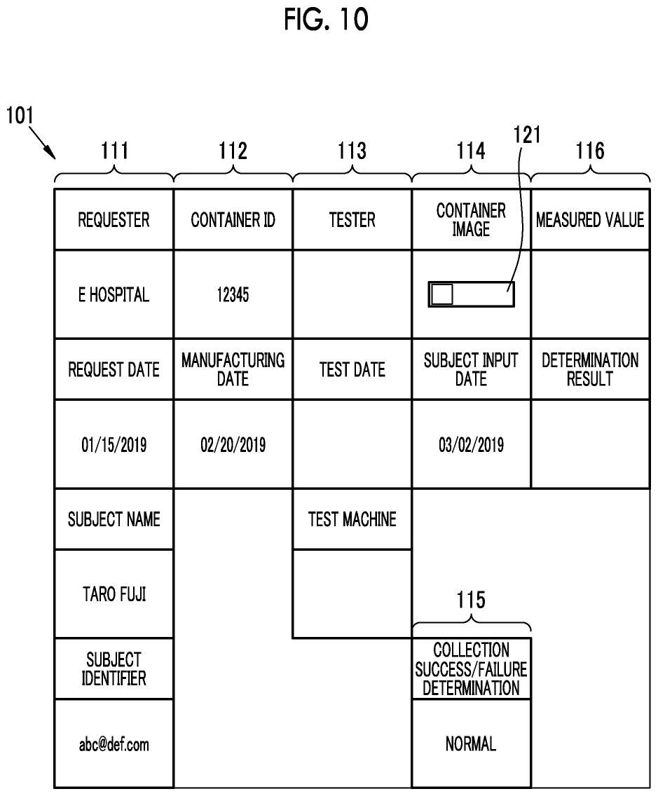

In a case where the management system 22 receives the subject identifier, the container information 112, and the container image 121 from the smartphone of the subject 10 (Step S404), the registration unit 65 performs a collation process with the database 61 by using the subject identifier (Step S405), and specifies the table 101 in which the received subject identifier is registered. Then, the registration unit 65 registers the “container ID” and the “manufacturing date”, which is the container information 112, and the “container image” and the “subject input date”, which is the subject input information 114, in the table 101 in which the received subject identifier is registered (Step S406). The subject input date is the reception date of the container image 121. In a case where the communication unit 63 receives the container image 121, the determination unit 64 determines the success or failure of the sample collection by using the container image 121 (Step S407), and the registration unit 65 registers the determination result related to the success or failure of the sample collection in the item of a “collection success/failure determination” of the specified table 101. At this stage, as shown in FIG. 10, in the table 101, the container information 112, the subject input information 114, and the determination result 115 of sample collection success/failure are registered in association with the test order 111.

In a case where the determination result 115 of sample collection success/failure is “abnormal”, the management system 22 sends, to the smartphone, which is the subject terminal, a notification such as a message (email) confirming whether or not the stool collection (sample collection) has been performed and/or a message (email) prompting the sample collection again, by using the communication unit 63. Upon receiving this notification (Step S409), the subject 10 can perform necessary procedures such as the procedure for resending the sample container 13. As a result, it is possible to prevent the sample container 13 where the sample may not be correctly collected or stored from being submitted to the hospital 11 or the like, and the test workflow can be smoothly advanced as compared with the case where the notification is not given. In a case where the determination result 115 of sample collection success/failure is “normal”, the subject 10 submits the sample container 13 to the hospital 11 or the like (Step S410).

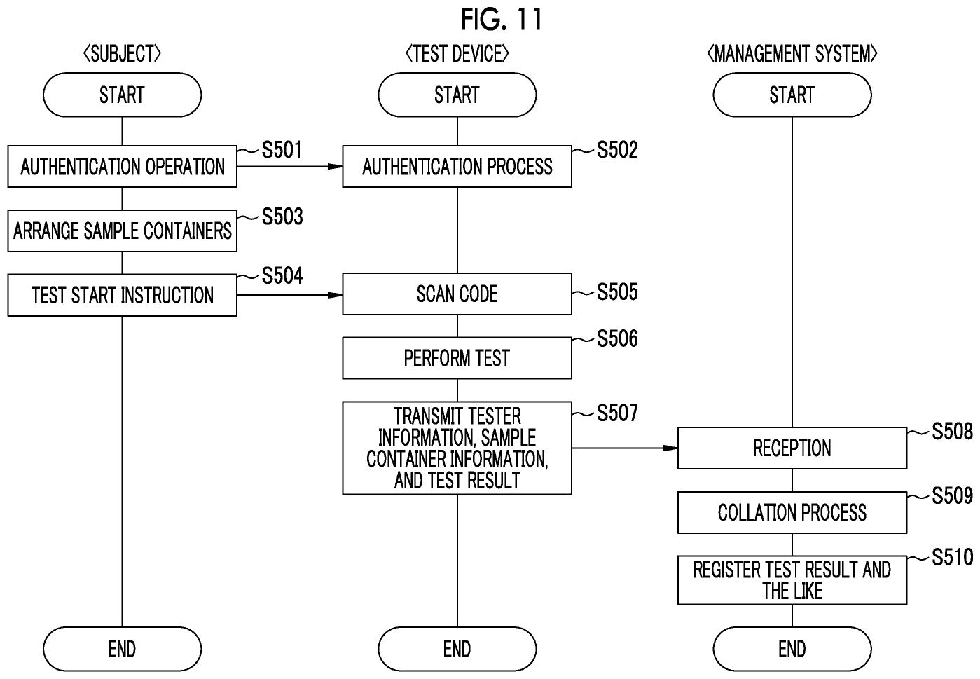

The management system 22 acquires and registers the tester information (Step S104) and acquires and registers the test result (Step S105) as follows. That is, as shown in FIG. 11, in a case where the testing institution 12 receives the sample container 13, the tester performs an authentication operation for use of the test device 21 to be used (Step S501). The test device 21 receives the authentication operation and performs an authentication process (Step S502) to obtain information such as the name of the tester, which is one of the tester information 113. Further, in a case where the authentication operation of the test device 21 is completed, the tester arranges one or a plurality of sample containers 13 in the test device 21 (Step S503), and inputs a test start instruction (Step S504).

Upon receiving the test start instruction, the test device 21 scans the code 39 of the sample container 13 to acquire the container information 112 (Step S505). Then, the test device 21 performs the test on the sample held by the sample container 13 for which the container information 112 is obtained (Step S506). After that, in a case where the measurement result is obtained, the test device 21 transmits the container information 112, the tester information 113, and the test result 116 to the management system 22 (Step S507).

In the management system 22, in a case where the communication unit 63 receives the tester information 113 and the test result 116 (Step S508), the registration unit 65 performs the collation process with the database 61 by using the container information 112 (Step S509), and specifies the table 101 in which the received container information 112 is registered. Then, the registration unit 65 registers the tester information 113 and the test result 116 in the table 101 in which the received container information 112 is registered (Step S510). Thereby, the table 101 in which the test order 111, the container information 112, the tester information 113, the subject input information 114, the determination result 115 of sample collection success/failure, and the test result 116 are associated with each other is completed (see FIG. 5).

As described above, the management system 22 automatically associates the test order 111, the container information 112, the tester information 113, the subject input information 114, the determination result 115 of sample collection success/failure, and the test result 116. That is, according to the management system 22, the tester does not have to make these associations at the testing institution 12. Therefore, the management system 22 can reduce the burden of the association of the tester at the testing institution 12 as compared with the test system or the like in the related art in which the tester has to manually associate the information.

Further, according to the management system 22, it is possible to register and manage that the sample is correctly collected and stored before the test is performed. Therefore, according to the management system 22, it is possible to prevent the sample container 13 in which the sample is not correctly collected and stored from being submitted, or to know that there is a sample container 13 in which the sample is not correctly collected and stored before the test is performed. Therefore, the management system 22 can smoothly proceed with the test and the registration of the test results, as compared with the test system or the like in the related art which does not manage the correct collection and storage of the sample by registration or the like.

Further, by registering and managing the data as described above in the management system 22, even in a case where a plurality of sample containers 13 are set at the same time in the test device 21, it is not necessary to give special consideration to the arrangement order of these sample containers 13. Therefore, as compared with the test system in the related art in which the sample container 13 is specified by the arrangement order of the sample containers 13 in the test device 21, the introduction of the management system 22 has an advantage that the test can be easily performed.

In the fecal occult blood test, the subject 10 stores and submits two days' worth of samples in separate sample containers 13. That is, in the fecal occult blood test, one subject 10 submits two sample containers 13. Therefore, the registration unit 65 creates two tables 101 for one subject 10, and registers the sample container 13 used on the first day and the sample container 13 used on the second day in each table 101. For example, in a case where the code 39 is divided into the color for the first day (for example, blue) and the color for the second day (for example, red), by these differences in color, the sample container 13 used on the first day and the sample container 13 used on the second day can be discriminated. Of course, in a case where data for distinguishing between the first day and the second day is given to the code 39 as one of the container information 112, the above color division is not necessary.

Further, in addition to the two-dimensional code and the like, the code 39 may be given a discrimination code (not shown) for identifying between the first day and the second day. In a case where the sample container 13 on the first day and the sample container 13 on the second day are discriminated by using the container image 121, the discrimination can be performed by, for example, the determination unit 64. Of course, a discrimination unit (not shown) for making the discrimination may be provided. Further, in a case of scanning the code 39, it is possible to discriminate between the sample container 13 on the first day and the sample container 13 on the second day. In this case, the container information 112 can include information indicating whether the sample container 13 is the sample container 13 on the first day or the sample container 13 on the second day, incidental to the scan result of the code 39. In this case, the management system 22 registers and manages whether the sample container 13 is for the first day or the second day based on the container information 112. As described above, in a case of registering and managing the sample containers 13 for two days, it is possible to reliably prevent problems such as submitting only the sample for one day and submitting the sample container 13 without collecting the sample for the second day.

In the fecal occult blood test, the subject 10 may attempt to capture and submit both the sample container 13 on the first day and the sample container 13 on the second day in one container image 121. Therefore, in a case where a plurality of sample containers 13 are included in one container image 121, it is preferable to provide an image dividing portion (not shown) that divides these into the container image 121 for each sample container 13. In a case where the image dividing portion is provided, the management system 22 can continue the process normally even in a case where the subject 10 stores the sample containers 13 for two days in one container image 121 and transmits the image.

In the above embodiment and the like, only the code 39 is attached to the sample container 13, but as shown in (A) and (B) of FIG. 12, it is preferable to attach, for example, a white sticker 151 to one side of the transparent portion of the sample container 13 (particularly the portion used by the determination unit 64 for determination). Since the subject 10 captures the container image 121 in which the sample container 13 is captured, the environment in which the sample container 13 is placed is not constant at the time of capturing an image. For example, in a case where the sample container 13 is placed on a brown table and the container image 121 is captured, there may be a problem that the storage chamber 34 turns brown, reflecting the brown color of the table. Therefore, in a case where the subject 10 captures an image of the front surface to which the code 39 is attached as shown in (A) of FIG. 12, the sticker 151 is attached to the back surface of the sample container 13 to which the code 39 is not attached as shown in (B) of FIG. 12. In this way, even though the container image 121 for the sample container 13 is captured on a table of any color, the container image 121 having the color of the sticker 151 as the background can be obtained. As described above, the sticker 151 determines the background color of the container image 121. Therefore, the color of the sticker 151 (particularly the color of the surface to be attached to the sample container 13) does not necessarily have to be white as long as it is common to the plurality of sample containers 13. Here, the color of the sticker 151 (the color of the surface to be attached to the sample container 13) is preferably a color that can correctly identify the sample or the storage solution 33 for storing the sample, such as being a complementary color to the sample.

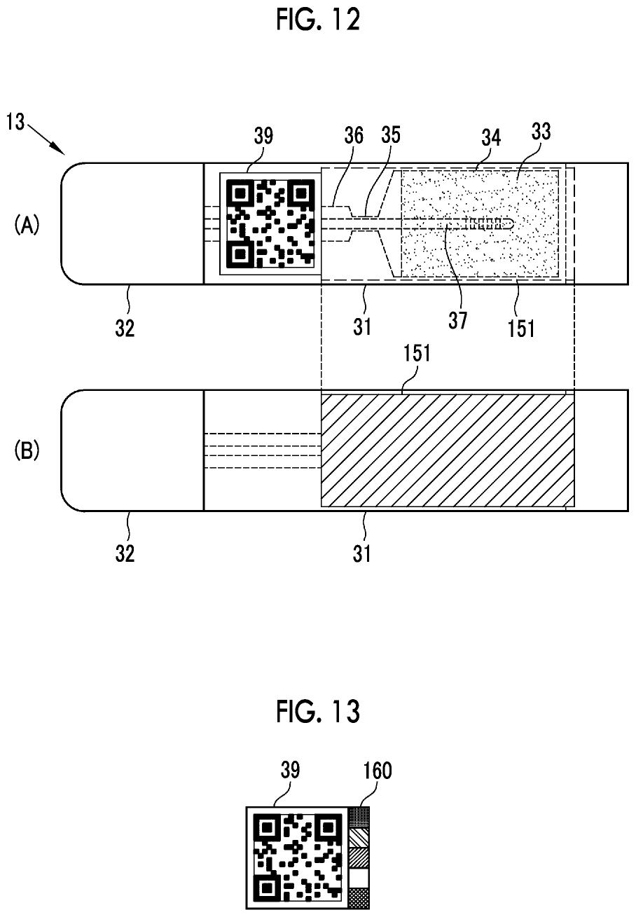

Normally, the place where the sample container 13 is imaged is indoors, and the color of the container image 121 changes depending on the type of the indoor lighting device (fluorescent lamp, incandescent lamp, or the like). Thus, the determination unit 64 needs to use the color of the storage chamber 34 for determining the sample collection success/failure in consideration of its influence. For example, in a case where the sample container 13 is imaged under an incandescent lamp, the container image 121 has a yellowish color. Therefore, in a case where the determination unit 64 does not consider the lighting environment, the determination unit 64 may determine the sample collection as “normal” even in a case where the collection is not actually performed correctly. Therefore, as shown in FIG. 13, for example, a color standard pattern 160 for color calibration may be provided adjacent to the code 39. The color standard pattern 160 is composed of, for example, five colors of red, green, blue, white, and black, and the storage chamber 34 and the color standard pattern 160 are imaged together. Then, the determination unit 64 determines the sample collection success/failure after correcting the color change (effect) caused by the lighting environment by using the color of the color standard pattern 160 in the container image 121. In this way, in a case where the color standard pattern 160 is provided and the sample collection success/failure determination is performed using the color standard pattern 160 in the container image 121, it is possible to accurately determine the sample collection success/failure regardless of the environment in which the sample container 13 is imaged. Further, the sample collection success/failure is determined by the color of the storage chamber 34, but in the determination of the sample collection success/failure, the presence or absence of solid components of feces in the storage chamber 34 can be considered instead of the color of the storage chamber 34, or in addition to the color of the storage chamber 34.

In the above embodiment and the like, the information of the “manufacturing date” of the sample container 13 and the information of the “subject input date” and the “test date” are registered in the table 101. On the other hand, the sample container 13 usually has an expiration date for guaranteeing quality and the like. Therefore, the management system 22 preferably compares the “manufacturing date” of the sample container 13 with the “subject input date” or the “test date” to check whether the sample container 13 is within the expiration date. This is to prevent inaccurate test results 116 from being obtained by using the sample container 13 that has exceeded the expiration date. The expiration date of the sample container 13 can be checked by, for example, the control unit 66 based on the registration information in the table 101. Of course, apart from the control unit 66 and the like, an expiration date check unit for confirming the expiration date of the sample container 13 may be provided. Since the “subject input date” is substantially the collection date of the sample, by comparing the subject input date with the “manufacturing date” of the sample container 13, it is possible to check whether or not the sample container 13 is within the expiration date at the time of collecting the sample, and it is possible to prevent the submission of the sample using the sample container 13 that has exceeded the expiration date. In a case where the sample container 13 has exceeded the expiration date at the time of collecting the sample, it is preferable to notify about the fact and prompt the sample collection again. Further, the check of the expiration date by comparing the “test date” and the “manufacturing date” of the sample container 13 can confirm whether or not the sample is appropriately stored on the date at which the test is performed.

In addition, the users of the management system 22 and the management system 22 can grasp the number of days elapsed from the sample collection (“subject input date”) to the test (“test date”) (the elapsed date and time in the case of registering up to the time). In addition, the users of the management system 22 and the management system 22 can grasp the collection timing of the sample from the “subject input date”. For example, it is possible to grasp whether the stool was collected in the morning or in the evening.

In the above embodiment and the like, in a case where the determination result 115 of the success or failure of the sample collection is “abnormal”, the subject 10 is notified to that effect, but in a case where the determination result 115 of the success or failure of the sample collection is “abnormal”, the management system 22 can notify the administrator of the management system 22, the employees of the testing institution 12, the related persons of the hospital 11, or the like to that effect instead of the notification to the subject 10, or together with the notification to the subject 10. In this case, the subject 10 can redo the collection of the sample by receiving appropriate guidance or the like from the related persons of the hospital 11.

In the above embodiment and the like, in a case where the management system 22 displays a part or all of the information registered in the table 101 or the table 101 on the display unit 67, it is preferable to display at least the container image 121. This is because the container image 121 can be presented to a tester or the like to provide an opportunity to confirm the determination result of the determination unit 64. The container image 121 may be displayed in a case where a predetermined mark or the like is pressed.

In the above embodiment and the like, the determination unit 64 determines the success or failure of the sample collection, but in addition to this, the determination unit 64 can determine the state of the sample by using the container image 121. For example, the determination unit can determine whether the sample stool is loose stool or not.

The management system 22 in the above embodiment and the like can use tests other than the fecal occult blood test, specifically, tests such as urinalysis, blood test, or genetic test at the testing institution 12 and the like. In a case where the test to be performed is a blood test, the determination unit 64 determines the presence or absence of hemolysis.

The management system 22 in the above embodiment and the like can acquire data such as X-ray images, ultrasonic images, and/or findings about these images from the hospital information system 16 and the like, aggregate them with the test results 116 and the like, and notify the subject 10 or the like.

In the above embodiment and the like, the subject 10 obtains the sample container 13 from the testing institution 12 or the hospital 11, but the subject 10 may be able to obtain the sample container 13 from a place other than the testing institution 12 or the hospital 11. For example, there is a case where the subject 10 optionally decides to carry out the test and purchases the sample container 13 through Internet sales or the like. In this case, the testing institution 12 cannot create the table 101 for a test because there is no test order 111. Therefore, the code 39 of the sample container 13 that the subject 10 independently obtains without going through the testing institution 12 or the like preferably includes at least a uniform resource locator (URL) indicating a website for registering the subject identifier in the management system 22, or a control command for displaying the website. Even in a case where the sample container 13 independently obtained by the subject 10 is used, the testing institution 12 can smoothly perform the test.

In addition to the control command and the like in the above embodiment, the code 39 can include a URL or a control command that guides the user to a website that guides how to use the sample container 13. According to the website that guides how to use the sample container 13, the subject 10 can smoothly transmit the container image 121 and the like. In particular, in a case where the imaging range of the sample container 13 is guided on the website that guides how to use the sample container 13, the registration of the container image 121 that cannot be used for the determination in the determination unit 64 can be reduced.

As described above, in a case of guiding how to use the sample container 13 through scanning the code 39, it is preferable that the subject 10 uses a smart speaker or other electronic devices that emit a voice as the subject terminal. This is because the subject 10 can receive voice guidance, free both hands, and accurately collect a sample and the like. In addition, it is not necessary to hold or lean against a smartphone or the like. Further, it is preferable that the smart speaker or the like is provided with a display unit. This is because an image, a moving image, or the like can be displayed to guide the subject 10 in an easy-to-understand manner such as how to use the sample container 13. In addition, the subject 10 can use a plurality of subject terminals in a complex manner. For example, in a case where the container image 121 is imaged by the camera of a smartphone, the subject 10 can receive guidance on the procedure, the imaging range, and the like from the smart speaker.

In each of the above embodiments and modification example, hardware structures of the processing units that execute various processes such as the database 61, the test order acquisition unit 62, the communication unit 63, the determination unit 64, the registration unit 65, and the control unit 66 are various processors as shown below. The various processors include a central processing unit (CPU) that is a general-purpose processor that functions as various processing units by executing software (program), a programmable logic device (PLD) that is a processor whose circuit configuration can be changed after manufacture, such as a graphical processing unit (GPU) or a field programmable gate array (FPGA), a dedicated electrical circuit that is a processor having a circuit configuration designed exclusively for executing various types of processing, and the like.

One processing unit may be configured by one of various processors, or may be configured by a combination of the same or different kinds of two or more processors (for example, a combination of a plurality of FPGAs, a combination of a CPU and an FPGA, a combination of a CPU and a GPU, or the like). In addition, a plurality of processing units may be configured by one processor. As an example of configuring a plurality of processing units by one processor, first, as represented by a computer, such as a client or a server, there is a form in which one processor is configured by a combination of one or more CPUs and software and this processor functions as a plurality of processing units. Second, as represented by a system on chip (SoC) or the like, there is a form of using a processor for realizing the function of the entire system including a plurality of processing units with one integrated circuit (IC) chip. Thus, various processing units are configured by using one or more of the above-described various processors as hardware structures.

More specifically, the hardware structure of these various processors is an electrical circuit (circuitry) in the form of a combination of circuit elements, such as semiconductor elements.

### EXPLANATION OF REFERENCES

10: subject

11: hospital

12: testing institution

13: sample container

14: manufacturer

16: hospital information system

21: test device

22: management system

26: manufacturing device

27: code creating unit

28: code attaching unit

31: container body

32: cap

33: storage solution

34: storage chamber

35: scraping portion

36: insertion path

37: stool collection rod

39: code

41: sample container placing portion

42: test unit

43: touch panel

51: code scanner

53: fecal occult blood test mechanism

61: database

62: test order acquisition unit

63: communication unit

64: determination unit

65: registration unit

66: control unit

67: display unit

68: operation unit

69: code scanner

101: table

111: test order

112: container information

113: tester information

114: subject input information

115: determination result

116: test result

121: container image

151: sticker

160: color standard pattern

A1 to A8: steps related to test order and the like

S101 to S510: steps related to operation of management system

What is claimed is:

1. A management system comprising:
a test order acquisition unit configured to acquire a test order including a subject identifier that specifies a subject terminal used by a subject;
a communication unit configured to receive the subject identifier, container information, and a container image from the subject terminal through communication with the subject terminal, the container information being information related to a sample container used by the subject for storing a sample, and the container image being an image of the sample container;
a determination unit configured to determine a success or failure of sample collection by using the container image; and
a registration unit configured to register the test order, the container information, and a determination result in the determination unit in association with each other by using the subject identifier.

2. The management system according to claim 1, wherein the subject identifier is an e-mail address or a telephone number used by the subject.

3. The management system according to claim 1, wherein the container information includes at least a container identifier that specifies the sample container.

4. The management system according to claim 1,
wherein the sample container has a transparent portion in which the sample is stored and/or a transparent portion through which the sample passes, and
the container image is an image including the transparent portion of the sample container.

5. The management system according to claim 4, wherein the determination unit determines the success or failure of the sample collection based on a color and/or a density of the transparent portion.

6. The management system according to claim 4, wherein the determination unit determines that the collection of the sample is successful in a case where the sample or a portion containing the sample has a specific color and a density equal to or higher than a specific density, and determines that the collection of the sample fails in a case where the sample or the portion containing the sample does not have the specific color or in a case where the sample or the portion containing the sample has a density lower than the specific density.

7. The management system according to claim 1,
wherein the sample container has a two-dimensional code including the container information, and
the subject terminal acquires the container information from the two-dimensional code and transmits the container information to the communication unit.

8. The management system according to claim 7, wherein the two-dimensional code includes a control command for controlling the subject terminal.

9. The management system according to claim 8, wherein the control command includes an information transmission command for transmitting the container information and the container image to the communication unit.

10. The management system according to claim 8, wherein the control command includes a camera activation command for activating a camera that captures the container image.

11. The management system according to claim 1,
wherein the communication unit receives the container information and a test result from a test device that tests the sample, and
the registration unit registers the test result in association with the test order by using the container information.

12. A management system comprising a processor, wherein the processor:
acquires a test order including a subject identifier that specifies a subject terminal used by a subject;
receives the subject identifier, container information, and a container image from the subject terminal through communication with the subject terminal, the container information being information related to a sample container used by the subject for storing a sample, and the container image being an image of the sample container;
determines a success or failure of sample collection by using the container image; and
registers the test order, the container information, and a determination result in the determination unit in association with each other by using the subject identifier.
